(19) United States
(12) Patent Application Publication
(10) Pub. No.: US 2023/0117827 A1
(43) Pub. Date: Apr. 20, 2023

(54) COMPOSITION COMPRISING A FLUOROPHORE LABELLED UPAR-TARGETING PEPTIDE CONJUGATE

(71) Applicant: FLUOGUIDE A/S, COPENHAGEN N (DK)

(72) Inventors: Grethe Nørskov Rasmussen, FARUM (DK); Andreas Kjær, FREDERIKSBERG (DK); Morten Albrechtsen, CHARLOTTENLUND (DK); Line Hartvig, HØRSHOLM (DK)

(73) Assignee: FLUOGUIDE A/S, COPENHAGEN N (DK)

(21) Appl. No.: 17/792,336

(22) PCT Filed: Jan. 15, 2021
(86) PCT No.: PCT/EP2021/050857

§ 371 Date: Jul. 12, 2022

(30) Foreign Application Priority Data
Jan. 17, 2020 (SE) 2050040-1

(51) Int. Cl. A61K 49/00

(52) U.S. Cl. CPC A61K 49/0056; A61K 49/0034

(57) ABSTRACT

The present invention describes a composition comprising a fluorophore labelled uPAR-targeting component, a buffer and a surfactant, wherein the fluorophore labelled uPAR-targeting component is solubilized in the composition by means of the surfactant being present, and wherein the composition comprises a maximum of 10 wt % water, preferably a maximum of 5 wt % water.

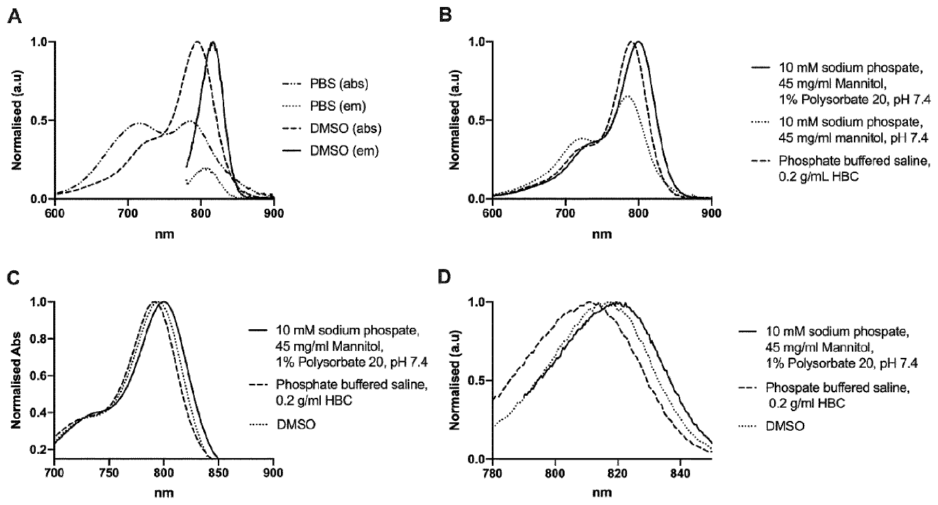

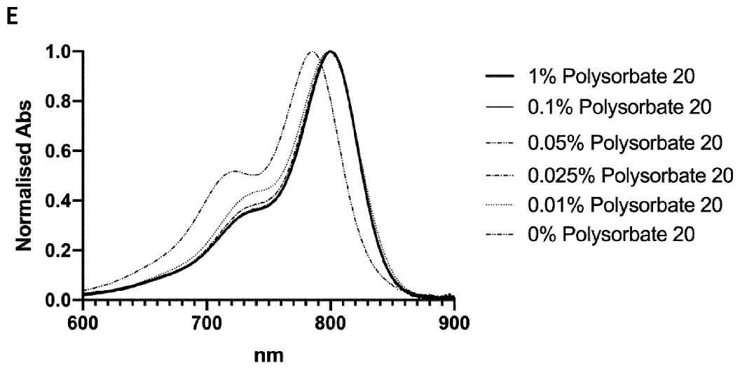

### FIELD OF THE INVENTION

The present invention relates to a composition comprising a fluorophore labelled receptor-targeting component, preferably a fluorophore labelled receptor-targeting peptide conjugate, more preferably a fluorophore labelled uPAR (urokinase Plasminogen Activator Receptor)-targeting peptide conjugate.

### TECHNICAL BACKGROUND

There are existing compositions comprising one or more fluorophore labelled receptor-targeting peptide conjugates where the receptor is uPAR. For example, in WO2016/041558 there is disclosed a conjugate that binds to the cell surface receptor uPA (uPAR). The conjugate is based on a fluorescence-labeled peptide useful as a diagnostic probe to the surfaces of cells expressing uPAR. The conjugate is capable of carrying a suitable detectable and imageable label that will allow qualitative detection and also quantitation of uPAR levels in vitro and in vivo. This renders the surgical resection of tumors more optimal. Furthermore, different alternatives of the conjugate are described in WO2016/041558, e.g. a conjugate comprising ICG-Glu-Glu-AE105 where ICG is indo-cyanin green, Glu-Glu is two glutamic acids acting as linkers and AE105 is an uPAR targeted peptide.

The present invention is directed to providing a formulation comprising a fluorophore labelled uPAR-targeting peptide conjugate, e.g. ICG-Glu-Glu-AE105 or other alternatives. The formulation according to the present invention provides for improved solubility of the fluorophore labelled uPAR-targeting peptide conjugate as well as increased stability for the entire formulation and fluorophore labelled uPAR-targeting peptide conjugate comprised therein.

### SUMMARY OF THE INVENTION

The latter stated purpose above is achieved by a composition comprising a fluorophore labelled receptor-targeting component, a buffer and a surfactant, wherein the fluorophore labelled receptor-targeting component is solubilized in the composition by means of the surfactant being present, and wherein the composition comprises a maximum of 10 wt % water, preferably a maximum of 5 wt % water. As should be understood from above, the present invention is particularly directed to a composition comprising a fluorophore labelled receptor-targeting peptide conjugate, more particularly a fluorophore labelled uPAR-targeting peptide conjugate, such as a peptide conjugate with a sequence homology of 80% to the peptides shown in table 4 in “Peptide-Derived Antagonists of the Urokinase Receptor. Affinity Maturation by Combinatorial Chemistry, Identification of Functional Epitopes, and Inhibitory Effect on Cancer Cell Intravasation” in Biochemstry 2001, 40, 12157-12168, Michael Ploug et al. It should, however, be noted that with a receptor-targeting component this may include a small molecule, protein, antibody, frab or of course a peptide, or any type of combination thereof.

The composition according to the present invention exhibits several advantages. The two most important are the full solubilizing of the fluorophore labelled receptor-targeting peptide conjugate and also a high stability of the entire composition. The stability is primarily driven by the lack of presence of water. For instance, a lyophilized product, which is one target product type according to the present invention, is one example where there is a low water content. However, to have a proper formulation, you should also have a product with a nice lyo cake appearance that easily solubilizes, hence the solubilizing of the fluorophore labelled receptor-targeting peptide conjugate is very important. Moreover, it should be noted that the composition according to the present invention, in different formulation forms, may have water level well below 5 wt %, for instance in the range of 1-3 wt %. In this regard it should also be noted that the water content may also increase during e.g. the 3 years shelf life.

As notable from above, the composition according to the present invention also comprises a buffer and a surfactant. The surfactant is the component ensuring that the fluorophore labelled receptor-targeting peptide conjugate is solubilized in the composition. Different types of surfactants and combinations thereof are possible to incorporate in the composition according to the present invention.

Furthermore, the buffer component may also be of different type according to the present invention. Some possible examples are provided below.

### Specific Embodiments of the Invention

Different aspects of the present invention are explained and handled further below. Moreover, some specific embodiments of the present invention are also described further.

The feature of the fluorophore labelled receptor-targeting component being solubilized in the composition can be seen or indicated in different ways in a composition according to the present invention. One such indication is when viewing the absorbance spectrum of the composition according to the present invention. This is further illustrated in the examples. Moreover, according to one specific embodiment of the present invention, the fluorophore labelled receptor-targeting component is solubilized in the composition at a level corresponding to a single peak with absorption maximum around 800 nm when measuring an absorbance spectrum of the composition in a wavelength area of 700-825 nm. It should be noted that the absorbance spectrum of the composition according to the present invention does not exclude more than one peak areas, such as a double peak, however the important feature in this regard is the high level of area in one predominant peak with absorption maximum around 800 nm. To give further clarification in this regard it may be said that an absorbance spectrum with a clear double peak is an indication that there is not a fully or substantially fully solubilization of the fluorophore labelled receptor-targeting component in the composition, and such a composition is not part of the scope of the present invention.

Furthermore, according to one specific embodiment of the present invention, the fluorophore labelled receptor-targeting component is solubilized in the composition at a level corresponding to having an absorbance spectrum peak with a maximum around 800 nm, and wherein the area of said absorbance spectrum peak is at least 50%, preferably at least 60%, even more preferably at least 65%, of the total area of the absorption spectrum in a given wavelength area of 600-900 nm.

This comparative area value of the single peak and the total area may be calculated by dividing the area in squares and determine the AUC (area under the curve) in the wavelength range of 600-900 nm. Then a line is drawn in the middle, that is at 750 nm. The AUCs on the left and right side, respectively, of the line at 750 nm are then calculated. Then the AUC on the right side, which is indicative for the single peak around 800 nm is divided with the total AUC calculated according to above. As said above, according to one embodiment this single peak around 800 nm provides for at least 50% of the total area in the wavelength range of 600-900 nm, preferably at least 60%, and more preferably above 65%.

The present invention suitably also comprises other components. For instance, a lyoprotectant is such a component. A lyoprotectant is a molecule which may be combined with a small molecule, peptide or protein and then significantly prevents or reduces chemical and/or physical instability of the small molecule, peptide or protein upon drying, particularly during lyophilization and subsequent storage. Some examples of lyoprotectants include sugars, e.g. sucrose or trehalose, amino acids, e.g. monosodium glutamate, histidine or arginine; methylamines, e.g. betaine; lyotropic salts, e.g. magnesium sulfate. Other examples include polyols, e.g. trihydric or higher sugar alcohols, e.g. glycerin, erythritol, glycerol, arabitol, xylitol, sorbitol, and mannitol. Yet some other examples are ethylene glycol; propylene glycol; polyethylene glycol; pluronics; or hydroxyalkyl starches, e.g. hydroxyethyl starch (HES). Also combinations are of course totally possible. According to one specific embodiment of the present invention, the composition comprises a lyoprotectant, preferably a lyoprotectant chosen from the group of sucrose, trehalose, mannitol, glycine or a combination thereof, more preferably mannitol or mannitol in combination with one or more other components, more preferably a combination of mannitol and glycine or a combination of mannitol and sucrose. Some of these preferred alternatives according to the present invention are further presented in the examples. Moreover, in this context it may also be said that it is typically suitable to use sufficient sugars to provide an isotonic formulation to avoid injection site reactions (stinging).

As described above, the composition according to the present invention comprises a surfactant. Many different forms of surfactants are possible to use according to the present invention. Without any limitation to possible alternatives according to the present invention, the following examples may be mentioned. According to one embodiment of the present invention, the composition comprises a non-ionic surfactant. One example is ethoxylates, such as fatty alcohol ethoxylates, alkylphenol ethoxylates (APEs), fatty acid ethoxylates, ethoxylated fatty esters and oils, or ethoxylated amines or fatty acid amines. Another type is fatty acid esters, e.g. fatty acid esters of polyhydroxy compounds, such as fatty acid esters of glycerol, sorbitol, sucrose or alkyl polyglucosides. Other examples are different types of amine oxides, polyoxamers, sulfoxides or phosphine oxides.

Moreover, different forms of tween surfactants are one specific example of interest according to the present invention. For instance, Tween 20 may be used, which is used in the alternatives presented in the examples. Tween 80 and other polysorbates are also totally possible to use instead. Furthermore, hydroxyl-beta-cyclodextrin is another possible example. Also combinations are of course possible to use.

It should be noted that many surfactant alternatives are possible according to the present invention, also ionic surfactants. Furthermore, different level of concentrations of the surfactants etc. are also fully possible according to the present invention, and of course not only the levels presented in the examples.

Another component included in the composition according to the present invention is at least one buffer/buffering agent. Also in this case many alternatives are totally possible. Non-limiting examples are different forms of borates, carbonates, citrates and phosphates, such as PBS. Moreover, glycine and tris and formulated tris solutions are also alternatives totally possible. Moreover, yet some other more specific examples are imidazole, succinic acid formulations. Also bicine formulations are possible, e.g. Bis-Tris or N,N-Bis(2-hydroxyethyl)glycine. Yet other specific examples are sulfonic acid formulations, such as 2-(N-Morpholino)ethanesulfonic acid or 4-Morpholineethanesulfonic acid, N,N-Bis(2-hydroxyethyl)-2-aminoethane-sulfonic acid, 3-(Cyclohexylamino)-1-propanesulfonic acid, 4-(2-Hydroxy-ethyl)piperazine-1-ethanesulfonic acid, N-(2-Hydroxyethyl)piperazine-N′-(2-ethanesulfonic acid).

According to one general embodiment of the present invention, the buffer is provided so that the composition has a physiological pH or substantially a physiological pH. As shown in the examples, for instance sodium phosphate to set a pH around 7.4 may be used according to the present invention.

Also the fluorophore may vary according to the present invention. First of all, according to one embodiment, the fluorophore labelled receptor-targeting component comprises a fluorophore, a peptide binding to the receptor and a linker group, wherein the fluorophore, the peptide binding to receptor and the linker group is connected by covalent bonds. As an example, the linker group may comprise oligoethylene glycols or other short oligomers such as oligo-glycerol, oligo-lactic acid or carbohydrates which are optionally connected by covalent bonds to at least one amino acid.

With reference to the fluorophore alternatives, many such are possible according to the present invention. According to one embodiment of the present invention, the fluorophore is preferably selected from any of indocyanin green (ICG), Methylene blue, 5-ALA, Protoporphyrin IX, IRDye800CW, ZW800-1, Cy5, Cy7, Cy5.5, Cy7.5, IRDye700DX, Alexa fluor 488, Fluorescein isothiocyanate, Flav7, CH1055, Q1, Q4, H1, IR-FEP, IR-BBEP, IR-E1, IR-FGP, or IR-FTAP, preferably wherein the fluorophore is indocyanin green (ICG).

Furthermore, the peptide involved may also be of different type. According to one specific embodiment, the peptide may be chosen from any of the following:

### -Asp-Cha-Phe-(D)Ser-(D)Arg-Tyr-Leu-Trp-Ser(-);

### -Asp-Cha-Phe-(D)Ser-(D)Arg-Tyr-Leu-Trp-Ser-OH; or

### -Asp-Cha-Phe-(D)Ser-(D)Arg-Tyr-Leu-Trp-Ser-NH₂.

Moreover, the amino acid may be selected from proteinogenic amino acids and non-proteinogenic amino acids, which includes natural amino acids and synthetic amino acids. In relation to this, it may further be mentioned that the natural amino acids may include C-alpha alkylated amino acids such aminoisobutyric acid (Aib), N-alkylated amino acids such as sarcosine, and naturally occurring beta-amino acids such as beta-alanine. Further, the synthetic amino acids may include amino acids with non-proteinogenic side-chains such as cyclohexyl alanine (Cha), gamma-amino acids, and dipeptide mimics. The term dipeptide mimics may be interpreted as an organic molecule that mimics a dipeptide by displaying the two amino acid side-chains, e.g., having a reduced amide bond linking two residues together. Amino acids with non-proteinogenic side-chains may also include amino acids with side-chains with restricted motion in chi-space. The term restricted motion in chi-space may be interpreted as restricted flexibility in the rotation of the side-chain groups. The oligopeptides may consist of up to fifty amino acids and may include dipeptides, tripeptides, tetrapeptides, and pentapeptides, and may further be made up by proteinogenic amino acids and non-proteinogenic amino acids.

In relation to the present invention it should be noted that when a peptide is included, not only AE105, is suitable. Many others, such as the ones disclosed in table 4 in “Peptide-Derived Antagonists of the Urokinase Receptor. Affinity Maturation by Combinatorial Chemistry, Identification of Functional Epitopes, and Inhibitory Effect on Cancer Cell Intravasation” in Biochemstry 2001, 40, 12157-12168, Michael Ploug et al., are also possible. Therefore, according to one specific embodiment of the present invention, the peptide is chosen from AE101, AE105, AE106, AE110, AE112, AE113, AE116, AE133, AE133*, AE134, AE135, AE136, AE137, AE138, AE139, AE145, AE140, AE141, AE142, AE143, AE144, AE164, AE164*, AE120, AE120* or AE151, wherein the following apply: AE101 is d-Cha-F-s-r-Y-L-W-S, AE105 is D-Cha-F-s-r-Y-L-W-S, AE106 is D-Cha-F-S-r-Y-L-W-S, AE110 is D-Cha-F-s-R-Y-L-W-S, AE112 is D-F-F-s-r-Y-L-W-S, AE113 is D-N-F-s-r-Y-L-W-S, AE116 is D-Cha-F-s-r-G-Y-L-W-S, AE133 is KGSGG-D-Cha-F-s-r-Y-L-W-S, AE133* is KGSGG-D-Cha-F-s-r-Y-L-W-S, AE134 is KGSGG-D-Cha-F-s-r-Y-L-W-A, AE135 is KGSGG-D-Cha-F-s-r-Y-L-A-S, AE136 is KGSGG-D-Cha-F-s-r-Y-A-W-S, AE137 is KGSGG-D-Cha-F-s-r-A-L-W-S, AE138 is KGSGG-D-Cha-F-s-a-Y-L-W-S, AE139 is KGSGG-D-Cha-F-a-r-Y-L-W-S, AE145 is KGSGG-D-Cha-F-A-r-Y-L-W-S, AE140 is KGSGG-D-Cha-A-s-r-Y-L-W-S, AE141 is KGSGG-D-A-F-s-r-Y-L-W-S, AE142 is KGSGG-A-Cha-F-s-r-Y-L-W-S, AE143 is KGSGG-D-Chp-F-s-r-Y-L-W-SC, AE144 is KGSGG-D-Cpa-F-s-r-Y-L-W-S^{C}, AE164 is KGSGG-D-F-F-s-r-Y-L-W-S, AE164* is KGSGG-D-F-F-s-r-Y-L-W-S, AE120 is [D-Cha-F-s-r-Y-L-W-S]2-/3A-K^{C}, AE120* is [D-Cha-F-s-r-Y-L-W-S]2-/3A-K^{c} and AE151 is [r-W-D-Cha-S-L-s-F-Y]2-/3A-K^{c}, or a combination thereof, or a peptide with a sequence homology of at least 80% to any of these peptides. According to one preferred embodiment, the peptide chosen is AE105.

Moreover, a “linker group” is a molecule that connects the two parts to create the conjugate and where their respective functions to a large degree is maintained, e.g. where a peptide binds to a receptor and a fluorophore lights up. The linker connects the two together (the peptide and the fluorophore) where their desired properties are preserved in total or partially. This means e.g. that the peptide still binds to the receptor and the fluorophore preserves its properties. A linker can be at least partly part of either of the two molecules linked. According to one embodiment, the linker is Glu-Glu.

According to one preferred embodiment of the present invention, the receptor is uPAR (urokinase Plasminogen Activator Receptor). Furthermore, according to yet another preferred embodiment, the fluorophore labelled receptor-targeting component is ICG-Glu-Glu-AE105:

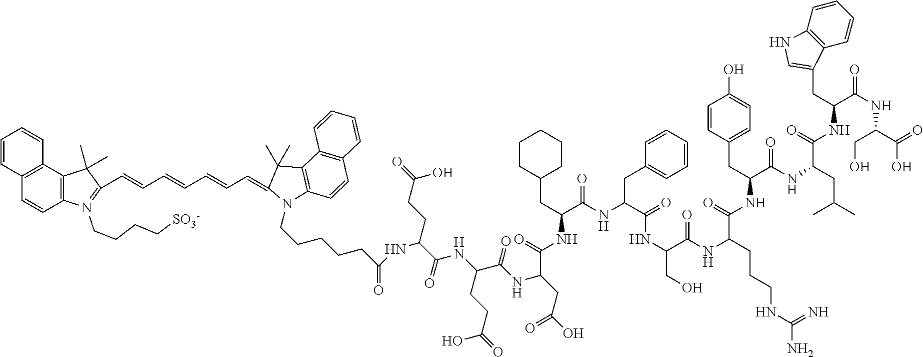

or a pharmaceutically acceptable salt thereof.

According to one embodiment, the concentration of ICG-Glu-Glu-AE105 is in the range of from 0.1-10.0 mg/ml, e.g. in the range of 0.1-8.0 mg/ml, e.g. in the range of 0.1-5.0 mg/ml, such as in the range of 0.2-3.0 mg/ml, preferably in the range of 0.5-2.0 mg/ml.

In line with the above, according to one specific embodiment of the present invention, the fluorophore labelled receptor-targeting component is ICG-Glu-Glu-AE105:

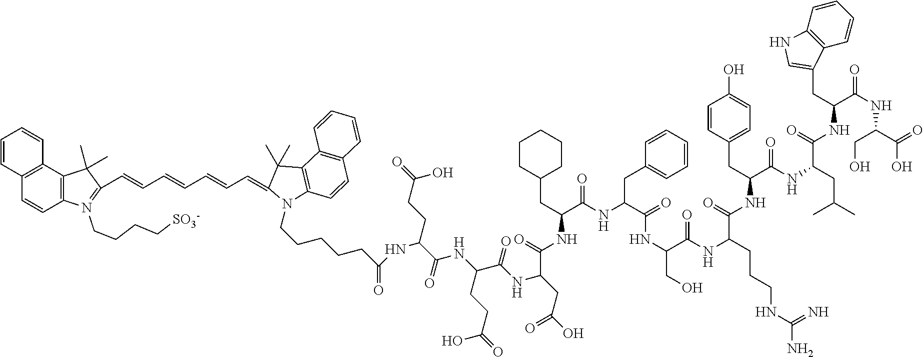

or a pharmaceutically acceptable salt thereof,
wherein the concentration of ICG-Glu-Glu-AE105 is in the range of from 0.1-10.0 mg/ml,
wherein the composition also comprises a buffer in the form of sodium phosphate in concentration of 5-50 mM,
and wherein the composition comprises a cryoprotectant combination of mannitol in a concentration of 10-50 mg/ml and glycine in a concentration of 1-30 mg/ml.

Furthermore, suitably the composition according to the present invention comprises at least one polysorbate, preferably Polysorbate 20, more preferably Polysorbate 20 in a concentration of >0.01 wt %.

The present invention also provides other aspects of interest for a composition comprising a fluorophore labelled receptor-targeting component. One such perspective is the pharmacokinetics profile. In line with this, according to one specific embodiment of the present invention, the fluorophore labelled receptor-targeting component comprises

a molecule binding to the receptor, preferably uPAR; and
a linker group which covalently links the fluorophore to the molecule binding to the receptor,
and wherein the conjugate is adapted to be administered systemically into a human or animal body.

Moreover, according to one embodiment, the fluorophore labelled receptor-targeting component has a pharmacokinetic profile where a TBR (tumor-to-background ratio) of at least 2.5 is reached within 3.5 hours post administration and where a level of TBR of at least 2.5 is held during at least 30 minutes before decreasing again, and wherein the fluorophore labelled receptor-targeting component is a human uPAR-targeting conjugate.

Furthermore, according to yet another embodiment, the fluorophore labelled receptor-targeting component has a pharmaco-kinetic profile where a TBR (tumor-to-background ratio) of at least 2.5 is reached within 3.5 hours post administration and where a level of TBR of at least 2.5 is held during at least 30 minutes before decreasing again, preferably wherein the fluorophore labelled receptor-targeting component is a human uPAR-targeting conjugate.

Moreover, according to yet another embodiment, the fluorophore labelled receptor-targeting component has a pharmacokinetic profile where the plasma half-life is maximum 75 hours, preferably maximum 20 hours, more preferably maximum 15 hours, more preferably in the range of 6-15 hours, most preferably in the range of 6-10 hours.

With reference to some expressions above and below, it may be mentioned that “blood” and “plasma” is sometimes used synonymously, however strictly speaking plasma is the yellowish liquid component of blood that normally holds the blood cells in whole blood in suspension. Plasma is the liquid part of the blood that carries cells and proteins throughout the body.

Also in this regard, the target receptor may be of different types according to the present invention. The targeting receptor may be urokinase Plasminogen Activator Receptor (uPAR), tissue factor (TF), epidermal growth factor receptor (EGFR), prostate-specific membrane antigen (PSMA), Vascular Endothelial Growth Factor (VEGF), Folate receptor, matrix metalloproteinase-2 (MMP-2), membrane type-I MMP, transmembrane inhibitor ofmetalloproteinase-2 (TIMP2), CIC-3 chloride ion channels, disaccharides and other glycans or glyco-phosphatidylinositol (GPI)-anchored cell membrane receptors. Furthermore, the receptor types may have a proteolytic activity or other enzymatic activity on the cell surface, such as e.g. urokinase (uPA). Furthermore, the receptor is such expressed in human cancer, e.g. such correlated with a poor prognosis, local invasiveness, or metastasis. In relation to this it may further be mentioned that the conjugate product according to the present invention is predominantly/partly anchored to the outside of the cells expressing the specific receptor, i.e. in contrast to the conjugate product being internalized into the cells expressing the receptor.

In relation to the receptor it may also be mentioned that when saying that the receptor (uPAR) is expressed on cancer cells this may also imply that they are expressed on the bodies ‘normal’ stroma cells influenced by cancer cells they are in contact with or in extremely close proximity to (e.g. 2-5 cells in between). This is also true for cases where the ‘normal’ cells help the cancer cells in invading normal tissue. For clarity ‘normal’ stroma cells in such close proximity is also included as cancer cells. Normal in quotes as it can be argued that the cells under influence by cancer cells and expressing uPAR may no longer be termed normal.

As hinted above, the receptor-targeting conjugate, suitably a uPAR-targeting conjugate, according to the present invention may also have an optimal receptor binding profile and pharmacokinetic profile. The uPAR-targeting conjugate injected systemically will distribute through the circulatory system to blood perfused tissues and organs in the body. For tissues with blood perfusion the uPAR-targeting conjugate will accumulate. When such tissue is exposed to light with a wavelength (color) being absorbed by the fluorophore contained in the uPAR-targeting conjugate, the fluorophore will emit light. The uPAR-targeting conjugate (‘L’ in the formula below) binds to the receptor (‘R’ in the formula below) according to first order kinetics:

R+L↔RL, and the reaction is characterized by the on-rate binding (Kₒₙ), the off-rate binding (Koff) and the resulting equilibrium binding constant K_{D}(K_{D}=Koff/Kon). Preferably, Kₒₙ>1×10³ M⁻¹s⁻¹ and/or K_{off}<1×10⁻¹ s⁻¹, more preferably Kₒₙ7.3×10⁵ M⁻¹ s⁻¹, as further mentioned below.

The product conjugate will distribute via the blood from where it will be eliminated via excretion by the liver, and/or the kidney, or by redistributes to compartments other than the blood. It results in the tissue with the presence of cells expressing the targeted receptor to where the uPAR-targeting conjugate is bounds will light up more than the background, creating the so called “TBR” (tumor-to-background ratio).The conjugate product is lighted up using a light source creating light of a specific wavelength and then detecting the light emitted from fluorophore using a specific filter for the specific emitted light. In a thought ideal situation, the cancer cells will light up immediately after injection, with sufficient high relative light intensity, without any background light, and the created desired TBR of at least 2.5 would last several hours. In a practical world it is acceptable if the desired TBR of 2.5 is reached within 3.5 hours, after injection and lasts at least 30 minutes. A further explanation with reference to this aspect and others are given below in relation to the present invention.

In line with the above, according to one embodiment of the present invention, the fluorophore labelled receptor-targeting component has a pharmacokinetic profile where a TBR (tumor-to-background ratio) of at least 2.8 is reached within 3.5 hours post administration and where a level of TBR of at least 2.8 is held during at least 30 minutes before decreasing again.

Furthermore, according to yet another embodiment, a peak TBR of the fluorophore labelled receptor-targeting component after administration is at least 3.

The conjugate product according to this embodiment of the present invention exhibits several features including some linked to its pharmacokinetic and receptor binding affinity. The receptor-targeting conjugate provides a specific combination of plasma half-life and receptor binding affinity.

In line with the above, according to one specific embodiment of the present invention the speed of which the protein (P)-ligand (L) complex takes place may be defined as

$$P + L {⇌}^{{K}_{on}} ,$$

where Kₒₙ is a constant of the binding reaction and where K_{off} is a constant for the dissociation of the protein-ligand complex, and wherein Kₒₙ>1×10³ M⁻¹s⁻¹ and/or K_{off}<1×10⁻¹ s⁻¹, more preferably wherein Kₒₙ≥7.3×10⁵ M⁻¹ s⁻¹.

Furthermore, according to yet another embodiment, Kₒₙ of the fluorophore labelled receptor-targeting component is equal to or higher than that of uPA being the natural ligand, implying Kₒₙ≥4.6×10⁶ M⁻¹ s⁻.

Moreover, according to yet another specific embodiment of the present invention, receptor binding affinity defined as IC₅₀ is a measurement of the ligand/receptor binding affinity. According to one embodiment, the fluorophore labelled receptor-targeting component displaces the natural ligand (uPA) binding to uPAR with an IC₅₀ value which is maximum 1,000 nM, preferably maximum 200 nM, more preferably maximum 50 nM, most preferably maximum 25 nM. Moreover, according to yet another embodiment of the present invention, receptor binding affinity of the fluorophore labelled receptor-targeting component to uPAR, defined as Kd, is maximum 2,500 nM, preferably maximum 2,000 nM, more preferably maximum 500 nM, most preferably in a range of 2,000-300 nM.

Below, several important aspects and features are further explained in relation to this aspect of the present invention. The conjugate composition should be administered systemically in a sufficiently high dose to allow sufficient distribution in the body to reach the targeted specific receptor present on a the cancer cells. This to ensure the conjugate binding to the target receptor quickly allowing a fast creation of the TBR in combination with a short plasma half-life and lasting for sufficiently long time to be useful. In short a high receptor binding affinity in combination with the short plasma-half-life creating a fast, high and long lasting TBR.

The TBR may be calculated from the relative intensity of light from tumor and background. The measurement of the light intensity may e.g. be performed using a simple commercially available camera with physical filters, such as, but not limited to, the clinically approved NIR-camera system Fluobeam®800 (Fluoptics, Grenoble, France) or EleVision™ (Medtronic, USA). Post image recording optimization of the image using software may be applied to enhance the TBR.

A high concentration will push the equilibrium towards more conjugate being bound to the target receptor. The concentration should however not be too high as this increases the risk of toxic effects for the patient, and the increases cost for the administration beyond what is practically acceptable. According to the present invention, the administration may be done systemically, preferably intravenously why the plasma concentration quickly reaches its highest concentration. The plasma concentration will decrease thereafter as the conjugate product is metabolized, excreted (by liver), eliminated (by kidney) and/or distributes to distribution compartment differently to the blood. According to the present invention, the concentration shall be sufficiently high and be maintained for a sufficiently long period for the conjugate product to reach a high enough and prolonged enough concentration in the compartment relevant for the receptor targeted (e.g. plasma, tissue stoma, cerebrospinal fluid, urine) for the conjugate product to bind to the receptors. According to one specific embodiment of the present invention, the dosing is performed in the range of 0.1-2,000 mg per dosage unit, preferably in the range of 1-1,000 mg per human dosage unit.

Furthermore, another important feature is the binding affinity to the target receptor, including the onset and the off-set, such as discussed above. This will mark the target tumor cells quickest possible, with the highest possible relative light intensity, highest possible contrast for the longest possible time. A fast onset binding and a slow offset are preferred. In relation to the above it may be mentioned that TBR is a feature measured in vivo and is created by a combination of several other features, such as plasma half-life, but where the receptor binding affinity is one important feature.

Moreover, also selectivity for cancer tissue is of interest in relation to the present invention. According to one specific embodiment of the present invention, the receptor-targeting conjugate has a selectivity for cancer tissue of at least 60%, preferably above 70%, more preferably above 80% and most preferably above 90%. Thus, the conjugate product is characterized by having a selectivity for cancer tissue on preferred at least 60%, or 70% or 80%, or 90%. With selectivity is understood the relative number of tissues samples removed by the surgeon he/she believes is cancer based on its light intensity/contrast and thereafter confirmed actually is cancer. An example is that the surgeon removed 10 tissues samples that he/she believes is cancer and seven of them is confirmed histologically is cancer given a selectivity of 7/1070%. If 100% of the tissue samples removed by the surgeon believing is cancer are proven to be cancer, the selectivity is 100%. If only half of the tissue samples removed by the surgeon is cancer and the other half is normal tissue the selectivity is 50%.

One other aspect in relation to the aspects above are the place of excretion and elimination. Different conjugate types according to the present invention excretes and/or eliminates in different organs and are as such not suitable for cancer types localized in these organs. Moreover, the type of indication and target receptor are also important according to the present invention. The preference here is that the receptor needs to be expressed on the cancer the patient has to the maximal benefit for the operator (e.g. the surgeon). Furthermore, it is of specific interest that the receptor is expressed on the right part of the cancer. This is of interest as normally the middle of the cancer is easy to see and remove by the surgeon. The borders and local invasive outgrowths from the cancer, however, are more difficult to see and separate from normal tissue by the surgeon, hence more difficult for the surgeon to remove and/or to save normal tissue.

Ideally the conjugate product is administered to the patient when the surgeon undertakes the surgical procedure of removing the cancer, including when the surgeon investigates the completeness of the surgical procedure by investigating the removed cancer tissue and by investigating if there are any cancer cells left in the patient right after having removed the cancer tissue, investigate the tissue removed or when planning the post-surgery treatment. This is e.g. right before or under the surgery, or right after, during or before the anesthesia. This is a huge improvement in comparison to other know alternatives which must be administered 6 hours, 12 hours, 18 hours or even 1-2 day in advance of surgery to be ready for use during surgery and not even in every case produce a satisfactory TBR or having a satisfactory specificity to cancer, meaning that no normal tissue is removed in the believe that it is cancer tissue. The combination of the features of receptor binding and plasma clearance according to the present invention will allow such improved use. In other words, the conjugate product according to the present invention has a pharmacokinetic profile that allows administration as near before the time of use for the surgeons as possible, e.g. around anesthesia. It may furthermore be said that the conjugate product according to the present invention enables that the time from administration to first feasible time for use is within at least 1200 minutes, such as within 600 minutes, e.g. within 300 minutes, or 120 minutes, such as preferably within 60 minutes, or even within 30 minutes, e.g. within 15 minutes from administration.

To summarize some different perspectives linked to the pharmacokinetic aspects of the present invention, the following may be stated. The conjugate according to the present invention suitably exhibit the following features:

A TBR is generated fast and lasts for a long time (during surgery) which is reached as a combination of:

reaching a sufficient high concentration in plasma;

reaching a sufficiently high concentration in the cancer tissue;

has appropriate binding kinetics;

has an appropriate plasma elimination half-life;

can be dosed at dose levels which are safe, causing no severe adverse events;

displays high selectivity to the receptor of interest that is extensively expressed on the cancer of interest, in the part of the cancer of interest and with a high selectivity to the cancer compared to normal tissue in near proximity to the cancer; and

is detectable with available / existing equipment.

The given features above may be measured and analyzed by different means and equipment.

In addition to an optimal pharmacokinetic profile, there are also other aspects of certain interest according to the present invention. Two such are the dissolvability and protein binding capacity. The composition according to the present invention provides preferable combinations of beneficial features. According to one specific embodiment, the composition is dissolvable in less than 10 minutes, preferably less than 5 minutes, more preferably less than 2 minutes, most preferably less than 1 minutes. Furthermore, according to yet another specific embodiment, the composition has protein binding in vivo which is greater than 50%, preferably greater than 75%, more preferably greater than 85%, more preferably greater than 90%, more preferably greater than 95%, most preferably greater than 99%.

As is evident from above, the conjugate and composition according to the present invention are intended to be used in cancer surgery, cancer therapy and/or cancer diagnosis. In line with this, according to one embodiment of the present invention, the receptor-targeting conjugate and composition according to the present invention are provided for use in cancer surgery, cancer therapy or diagnosis, such as for use in optical imaging/-fluorescence imaging (FLI) of cancer. It should be said that the conjugate and composition according to the present invention find use in several different types of indications. Some examples are glioblastoma, glioma, lung, colorectum, breast, prostate, stomach, gastric, liver, thyroid, bladder, esophagus, pancreas, kidney, corpus uteri, cervix uteri, melanoma, brain (incl. central and peripheral nervous system), ovary, gallbladder, head and neck (e.g. lip, oral cavity, larynx, nasopharynx, oropharynx, hypopharynx), multiple myeloma, testis, vulva, salivary glands, mesothelioma, penis, Kaposi sarcoma, vagina, neuroendocrine tumors, neuroendocrine carcinomas

In imaging there is of course also equipment present. The conjugate product and composition according to the present invention suitably contain a fluorescent chemical element that can re-emit light upon light excitation. The excitation and emitted light are specific to the fluorophore used. The excitation light is typically coming from a laser such as e.g. with a wavelength between 600 nm (nanometer) and 900 nm. The emitted light from the fluorophore is typically detected by a camera using a mechanical or software-based filter e.g. detecting light between 750 nm and 950 nm. The equipment used may be a surgical robot, surgical microscope, endoscope or a handheld device. The specification of the light source (e.g. the laser) and light detector (e.g. camera with filter) depends on the fluorophore chosen.

Several different types of procedures may be used according to the present invention. Non-limiting examples of surgical procedures are, day care surgery, open surgery, minimal invasive surgery and robot assisted surgery.

It can also be surgeries with different purpose. Non-limiting examples of surgical purposes are: Curative surgery (aims to remove all the cancerous tumor from the body—this is included into the marked size calculation), Preventive surgery (is used to remove tissue that does not contain cancerous cells but may develop into a malignant tumor, e.g. a polyps in the colon), Diagnostic surgery (helps to determine whether cells are cancerous, e.g. taking a biopsy with the aim of making a diagnostic or screenings test, such e.g. looking for colon rectal malignant polyps using a colorectal scope), Staging surgery (works to uncover the extent of cancer e.g. laparoscopy (a viewing tube with a lens or camera is inserted through a small incision to examine the inside of the body)), Debulking surgery (removes a portion, though not all, of a cancerous tumor. It is used in certain situations when removing an entire tumor may cause damage to an organ or the body), Palliative or supportive surgery (is used to treat cancer at advanced stages. It does not work to cure cancer, but to relieve discomfort or to correct other problems cancer or cancer treatment may have created. An example of supportive surgery is the insertion of a catheter to help with chemotherapy), Restorative surgery (is sometimes used as a follow-up to curative or other surgeries to change or restore a person's appearance or the function of a body part. E.g. following women with breast cancer), or Corrective surgery (is a reoperation to solve problems after surgery (or other treatments), e.g. bleedings or infection).

Another area of interest in relation to the present invention is photodynamic therapy. Photodynamic therapy (PDT) is increasingly being used as an attractive, alternative treatment modality for superficial cancer. The treatment comprises two relatively simple procedures: the administration of a photosensitive drug and illumination of the tumor to activate or heat the drug. The composition according to the present invention may be used in PDT treatment.

The present invention is also directed to a method intended for cancer therapy, staging or diagnosis, such as in optical imaging/fluorescence imaging (FLI) of cancer.

The method may be directed to a method which involves to diagnose an anatomical structure, guide the surgeon/robot, assist the surgeon/robot, increase survival, increase the amount of cancer tissue removed under surgery, increase the quality of life, reduce the amount of normal tissue removed, increase the certainty, reduce surgery time, improve the quality of surgery, improve the quality assurance of surgery, reduce the cost of surgery, improve the surgeon's performance, and/or improve the surgical outcome in any other way.

Furthermore, the present invention is also directed to a method involving optical imaging. Therefore, according to one specific embodiment of the present invention, there is provided an optical imaging method comprising the steps of:

(a) administering of a composition according to the present invention accumulating in a target tissue,
(b) illuminating the target tissue with light of a wavelength absorbable by the fluorophore; and
(c) detecting fluorescence emitted by the fluorophore and forming an optical image of the target tissue.

Furthermore, the composition according to the present invention may be provided in different forms. According to one specific embodiment of the present invention there is provided a lyophilized composition comprising the composition according to the present invention. In this case a lyoprotectant agent is suitable to generate a lyo cake that can easily be reconstituted.

Moreover, also dry formulations comprising the composition according to the present invention are of interest.

In general, and as hinted above, there are several aspects to consider when obtaining an optimal product according to the present invention. First of all, the pharmaceutical formulation should go into solution, and here the surfactant is important. As mentioned above, a more or less physiological pH should be provided by use of a buffer. The stability feature is of importance and this is provided by keeping the water level low and also by using certain excipients, if needed. Moreover, also lyo protectant components are of interest, such as e.g. one or more lyoprotectants.

Also the production method is relevant. According to one specific embodiment, the present invention is directed to a method for the production of a composition according to above, wherein said method comprises admixing the fluorophore labelled receptor-targeting component, the buffer, and the surfactant to solubilize the fluorophore labelled receptor-targeting component in the composition. As said above, preferably the receptor-targeting component is a uPAR-targeting peptide conjugate. Furthermore, according to one specific embodiment, a lyoprotectant is admixed with the composition so that a lyo cake is produced. Preferably, the lyo cake may easily be reconstituted. One suitable combination of a lyoprotectant according to the present invention is mannitol and glycin.

Furthermore, according to yet another specific embodiment of the present invention, the production method comprises

(i) mixing ICG-Glu-Glu-AE105:

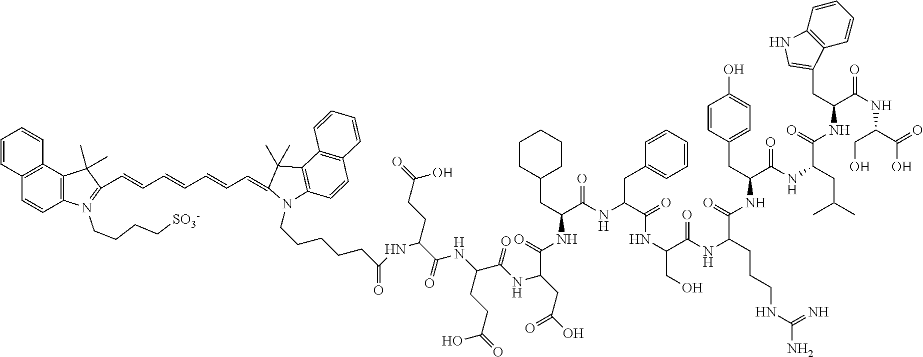

or a pharmaceutically acceptable salt thereof, with sodium phosphate, mannitol and glycine to yield a composition comprising

ICG-Glu-Glu-AE105 in a concentration of 0.1-10.0 mg/ml;

sodium phosphate in a concentration range of 5-50 mM;

mannitol in a concentration range of 10-50 mg/ml;

glycine in a concentration range of 1-30 mg/ml;

Polysorbate 20 in a concentration of >0.01 wt %.

(ii) adjusting the pH of the composition of step (i) to a pH in a range of 6.9-7.9;
(iii) transferring amounts of the mixture from step (ii) equivalent to the desired dosage into a suitable container;
(iv) drying the mixture; and
(v) sealing the container.

With reference to the above, a suitable concentration of the fluorophore labelled uPAR receptor-targeting component is in the range of 0.1-2,000 mg per dosage unit, preferably in the range of 1-1,000 mg per human dosage unit.

### EXAMPLES

### Example 1 Development of an Aqueous Formulation of a Composition According to the Present Invention

A composition comprising the fluorophore labelled receptor-targeting peptide conjugate ICG-Glu-Glu-AE105 (called composition 1 below) is soluble in DMSO in concentrations up to 20 mg/ml with a peak spectral absorption at 800 nm similar to indocyanine green (ICG) currently used in the clinic for visual assessment of vessels, blood flow and related tissue perfusion, fig. A.

An aqueous formulation of composition 1 of 1.0 mg/ml has been developed according to the present invention, using excipients suitable for pharmaceutical formulation development.

Composition 1 is not soluble in phosphate buffer saline at the tested concentration of 1.0 mg/ml, which can be observed both from the spectral absorption in the range from 700 nm to 825 nm with a clear double peak and the corresponding emission spectrum with a significantly lower fluorescent peak at 800 nm, fig. A. Excitation was conducted at 775 nm.

In a 10 mM sodium phosphate buffer containing 45 mg/ml mannitol, pH 7.4 the peak absorption shifted towards the peak absorption at 800 nm and in a formulation containing 10 mM sodium phosphate, 45 mg/ml mannitol, 1% Polysorbate 20, pH 7.4 the absorption spectrum was similar to the spectrum obtained in DMSO. Addition of 0.2 g/ml of 2-Hydroxypropyl-β-cyclodextrin (HBC) to phosphate buffer saline was also very efficient in solubilizing composition 1 in 1.0 mg/ml, fig. B.

The combined absorbance and emission spectra for the three formulations (1.0 mg/ml) with complete solubilization of composition 1 are presented in figs. C and D.

Further Explanation of the Figs. A-D
A. Absorbance and emission spectrum of FG001 dissolved in DMSO and PBS at 1.0 mg/ml. Excitation wavelength: 775 nm. Analytical samples for absorbance measurement were further diluted 200-fold and 400-1000-fold for emission spectra.
B. Absorbance spectrum of FG001 dissolved in 1) 10 mM sodium phosphate, 45 mg/ml mannitol, pH 7.4, 2) 10 mM sodium phosphate, 45 mg/ml mannitol, 1% Polysorbate 20, pH 7.4 and 3) PBS, 0.2 g/ml HBC. Analytical samples for absorbance measurement were further diluted 200-fold and 400-1000-fold for emission spectra
C. Overlay of absorption spectra for the three formulations (1.0 mg/ml) with complete solubilization of FG001: A) DMSO, B) 10 mM sodium phosphate, 45 mg/ml mannitol, 1% Polysorbate 20, pH 7.4 and C) phosphate buffer saline, 0.2 g/ml HBC. Analytical samples for absorbance measurement was further diluted 200-fold and 400-1000-fold for emission spectra
D. Overlay of emission spectra for the three formulations (1.0 mg/ml) with complete solubilization of composition 1: A) DMSO, B) 10 mM sodium phosphate, 45 mg/ml mannitol, 1% Polysorbate 20, pH 7.4 and C) phosphate buffer saline, 0.2 g/ml HBC. Excitation wavelength: 775 nm. Analytical samples for absorbance measurement were further diluted 200-fold and 400-1000-fold for emission spectra

### Example 2 Determination of the Optimal Concentration Range of Polysorbate 20 for Solubilization of Composition 1—See Fig. E

The optimal concentration range of Polysorbate 20 for solubilization of composition 1 at a concentration of 1.0 mg/ml in 10 mM sodium phosphate, 45 mg/ml mannitol, pH 7.4 was evaluated by measuring the absorption spectrum for formulations containing 0 to 1.0% Polysorbate 20.

The absorptions spectra showed that all formulations containing >0.01% Polysorbate 20, composition 1 was fully solubilized with the main peak spectral absorption at 800 nm.

### Example 3 Determination of the Stability of Composition 1 in Three Formulations with Polysorbate 20

The stability of composition 1 at a concentration of 1.0 mg/ml was evaluated in three different formulations suitable for development of a lyophilized product.

### A (Mannitol):

10 mM sodium phosphate, 45 mg/ml mannitol, 0.025% Polysorbate 20, pH 7.4

### B (Mannitol/glycine):

10 mM sodium phosphate, 26 mg/ml mannitol, 8.7 mg/ml glycine, 0.025% Polysorbate 20, pH 7.4

### C (Mannitol/sucrose):

10 mM sodium phosphate, 26 mg/ml mannitol, 40 mg/ml sucrose, 0.025%

### Polysorbate 20, pH 7.4

Composition 1 was weighed into the formulation buffer and pH was adjusted to 7.4 using sodium hydroxide or hydrochloric acid. The bulk formulation was sterile filtered through suitable sterile filters and filled into 6R vials. The vials were placed with stoppers in a lyophilizer and lyophilized using a standard program. Following lyophilization all vials were capped.

One vial of each formulation was reconstituted with water for injection prior to measuring osmolality and purity using RP-HPLC as described in example 4.

Osmolality and purity of composition 1 formulations following reconstitution

| | | Purity, main peak | Extra peak |
|---|---|---|---|
| | Osmolality | (area % | (area % |
| Formulation | (mOsmol/kg) | 780 nm) | 780 nm) |
| A: Mannitol | 289 | 94.3 | 1.1 |
| B: Mannitol/glycine | 283 | 95.4 | <0.1 |
| C: Mannitol/sucrose | 307 | 93.9 | 1.4 |

All three formulation had the desired osmolality for a pharmaceutical formulation to be used for intravenous administration to humans.

The stability of the reconstituted, liquid formulations of composition 1 was evaluated at room temperature and daylight (RTL) for two weeks.

| | | Purity, | |
|---|---|---|---|
| | | main peak | Extra peak |
| | Time | (area % | (area % |
| Formulation | (days) | 780 nm) | 780 nm) |
| A: Mannitol | 0 | 94.3 | 1.1 |
| | 7 | 93.5 | 1.9 |
| | 14 | 93.0 | 2.3 |
| B: Mannitol/ | 0 | 95.4 | <0.1 |
| glycine | 7 | 95.2 | 0.2 |
| | 14 | 95.1 | 0.1 |
| C: Mannitol/ | 0 | 93.9 | 1.4 |
| sucrose | 7 | 92.7 | 2.7 |
| | 14 | 92.7 | 2.6 |

It is well known that fluorophores like ICG are light sensitive and should be protected from light. However, to select the most stable formulation, the three liquid formulations were exposed to day light for two weeks at room temperature.

From the data presented in the table above, it is evident that formulation B: Mannitol/glycine is the most stable.

The stability of lyophilized product from the three formulations was also evaluated for two weeks at three different storage conditions:

### I: Room Temperature, Daylight (RTL)

### II: Room Temperature, Dark (RTD)

### III: 40° C., Dark (40D)

| | | Purity, | |
|---|---|---|---|
| | | main peak | Extra peak |
| | Storage | (area % | (area % |
| Formulation | condition | 780 nm) | 780 nm) |
| A: Mannitol | RTL | 95.0 | 0.3 |
| | RTD | NA* | NA |
| | 40D | 94.6 | 0.3 |
| B: Mannitol/ | RTL | 94.8 | 0.1 |
| glycine | RTD | 95.2 | 0.1 |
| | 40D | 94.8 | 0.1 |
| C: Mannitol/ | RTL | 94.7 | 0.3 |
| sucrose | RTD | 95.1 | 0.3 |
| | 40D | 94.7 | 0.2 |

*NA: Not sufficient lyophilized vials of formulation A

The stability of the lyophilized vials was significantly increased compared to the liquid formulation and smaller changes were observed. The study confirmed that Formulation B: Mannitol/glycine as lyophilizate has excellent stability both at elevated temperatures (40° C.) and upon exposure to light at room temperature.

The lyophilized samples stored at Room Temperature, Dark (RTD) and 40° C., Dark (40D) for two weeks were reconstituted and stored for an additional 24 hours at Room Temperature, Daylight (RTL).

| | | Purity, | |
|---|---|---|---|
| | | main peak | Extra peak |
| | Storage | (area % | (area % |
| Formulation | condition | 780 nm) | 780 nm) |
| A: Mannitol | 2 weeks lyo at RTD, | NA* | NA |
| | 24 hours liquid at RTL | | |
| | 2 weeks lyo at 40D, | 94.1 | 0.7 |
| | 24 hours liquid at RTL | | |
| B: Mannitol/ | 2 weeks lyo at RTD, | 94.7 | 0.1 |
| glycine | 24 hours liquid at RTL | | |
| | 2 weeks lyo at 40D, | 94.7 | 0.1 |
| | 24 hours liquid at RTL | | |
| C: Mannitol/ | 2 weeks lyo at RTD, | 93.9 | 0.9 |
| sucrose | 24 hours liquid at RTL | | |
| | 2 weeks lyo at 40D, | 93.9 | 0.8 |
| | 24 hours liquid at RTL | | |

*NA: Not sufficient lyophilized vials of formulation A

The stability of samples kept as a lyophilized product for two weeks and following reconstitution stored at room temperature, daylight for 24 hours also supports the selection of Formulation B: Mannitol/glycine as the preferred formulation.

It should be noted that another possible method to use to investigate the characteristic single absorbance spectrum peak is to calculate the single peak area positioned around 800 nm and to compare this with the total area of the absorption spectrum in a given wavelength area of 600-900 nm. This is further explained above in the description.

### Example 4 Determination of Long Term Storage Stability of Composition 1 in One Formulation with Polysorbate 20

The long term stability of composition 1 at a concentration of 1.0 mg/ml was evaluated as a lyophilizate in one formulation comprising 10 mM sodium phosphate, 26 mg/ml mannitol, 9.0 mg/ml glycine, 0.025% Polysorbate 20, pH 7.4 Composition 1 was weighed into the formulation buffer and pH was adjusted to 7.4 using sodium hydroxide or hydrochloric acid. The bulk formulation was sterile filtered through suitable sterile filters and filled into 6R vials. The vials were placed with stoppers in a lyophilizer and lyophilized using a standard program. Following lyophilization all vials were capped, visual inspected and stored protected from light at 5° C., 25° C./60% RH or 40° C./75% RH for up to 9 months

At the selected timepoint, a vial is removed from the stability chamber for visual inspection of the lyophilizate and water content determination. Another vial is reconstituted with water for injection prior to analyzing the corresponding liquid formulation for purity, pH and visual appearance.

Purity of reconstituted composition 1 formulations following storage at 5° C., 25° C./60% RH or 40° C./75% RH for up to 9 months

| | Purity, main peak | Purity, main peak | Purity, main peak |
|---|---|---|---|
| | (area % 780 nm) | (area % 780 nm) | (area % 780 nm) |
| Time | following storage | following storage | following storage |
| (months) | at 5° C. | at 25° C./60% RH | at 40° C./75% RH |
| 0 | 96.6 | 96.6 | 96.6 |
| 1 | 96.2 | 96.2 | 96.0 |
| 3 | 96.5 | 96.6 | 95.9 |
| 6 | 96.3 | 95.6 | 93.9 |
| 9 | 96.2 | 96.2 | Not analyzed |

Residual water, visual inspection before/after reconstitution and pH of composition 1 formulations following storage at 5° C. or 25° C./60% RH for up to 9 months.

| | Residual water, visual | | | Residual water, visual | | |
|---|---|---|---|---|---|---|
| | inspection and pH following | | | inspection and pH following | | |
| | storage at 5° C. | | | storage at 25° C./60% RH | | |
| | | Visual | | | Visual | |
| | | inspection | | | inspection | |
| | Residual | before/after | | Residual | before/after | |
| Time | water | recon- | | water | recon- | |
| (months) | (%) | stitution | pH | (%) | stitution | pH |
| 0 | 1.0 | Green | 7.4 | 1.0 | Green | 7.4 |
| 1 | 1.2 | solid/clear | 7.3 | 1.8 | solid/clear | 7.3 |
| 3 | 1.8 | green | 7.4 | 1.7 | green | 7.4 |
| 6 | 1.7 | solution | 7.3 | 1.7 | solution | 7.3 |
| 9 | 1.6 | | 7.4 | 1.9 | | 7.4 |

The selected formulation of composition 1 is shown to be stable for up to 9 months at storage conditions 5° C. and 25° C./60% RH judged by the purity determination. pH of the formulation as well as the appearance of the lyophilizate and the clarity of reconstituted liquid formulation remains unchaged for up to 9 months storage. Furthermore, the residual water content is around 2% following 9 months storage at 25° C./60% RH.

### Analytical Methods:

RP-HPLC to detect purity of reconstituted composition 1: Mobile phase A was composed of purified water/acetonitrile (80/20 v/v) with 5 mM ammonium acetate and mobile phase B was composed of purified water/acetonitrile (10/90 v/v) with 5 mM ammonium acetate. A Waters XBridge BEH Peptide, 3.5 μm, 130Å, 4.6×150 mm column was used. Flow rate was set to 1.0 mL/min,detection was at a wavelength of 780 nm, the column running temperature was 45° C. The sample cooler temperature was set at 5° C. and the sample injection load was 10 μg.

The water content of the lyophilizate was determined by Karl Fisher titration according to Ph. Eur. 2.5.32.

1. A composition comprising a fluorophore labelled receptor-targeting component, a buffer and a surfactant, wherein the fluorophore labelled receptor-targeting component is solubilized in the composition by means of the surfactant being present, and wherein the composition comprises a maximum of 10 wt % water, preferably a maximum of 5 wt % water.

2. The composition according to claim 1, wherein the composition comprises a fluorophore labelled receptor-targeting peptide conjugate.

3. The composition according to claim 1, wherein the fluorophore labelled receptor-targeting component is solubilized in the composition at a level corresponding to a single peak with absorption maximum around 800 nm when measuring an absorbance spectrum of the composition in a wavelength area of 700-825 nm.

4. The composition according to claim 1, wherein the fluorophore labelled receptor-targeting component is solubilized in the composition at a level corresponding to having an absorbance spectrum peak with a maximum around 800 nm, and wherein the area of said absorbance spectrum peak is at least 50%, preferably at least 60%, even more preferably at least 65%, of the total area of the absorption spectrum in a given wavelength area of 600-900 nm.

5. The composition according to claim 1, wherein the composition comprises a lyoprotectant, preferably a lyoprotectant chosen from the group of sucrose, trehalose, mannitol, glycine or a combination thereof, more preferably mannitol or mannitol in combination with one or more other components, more preferably a combination of mannitol and glycine or a combination of mannitol and sucrose.

6. The composition according to claim 1, wherein the composition comprises a non-ionic surfactant.

7. The composition according to claim 1, wherein the buffer is provided so that the composition has a physiological pH or substantially a physiological pH, preferably a pH in a range of 7.3-7.5.

8. The composition according to claim 1, wherein the fluorophore labelled receptor-targeting component comprises a fluorophore, a peptide binding to the receptor and a linker group, wherein the fluorophore, the peptide binding to receptor and the linker group is connected by covalent bonds.

9. The composition according to claim 8, wherein the fluorophore is selected from any of indocyanin green (ICG), Methylene blue, 5-ALA, Protoporphyrin IX, IRDye800CW, ZW800-1, Cy5, Cy7, Cy5.5, Cy7.5, IRDye700DX, Alexa fluor 488, Fluorescein isothiocyanate, Flav7, CH1055, Q1, Q4, H1, IR-FEP, IR-BBEP, IR-E1, IR-FGP, or IR-FTAP, preferably wherein the fluorophore is indocyanin green (ICG).

10. The composition according to claim 8, wherein the peptide is chosen from AE101, AE105, AE106, AE110, AE112, AE113, AE116, AE133, AE133*, AE134, AE135, AE136, AE137, AE138, AE139, AE145, AE140, AE141, AE142, AE143, AE144, AE164, AE164*, AE120, AE120* or AE151, wherein the following apply: AE101 is d-Cha-F-s-r-Y-L-W-S, AE105 is D-Cha-F-s-r-Y-L-W-S, AE106 is D-Cha-F-S-r-Y-L-W-S, AE110 is D-Cha-F-s-R-Y-L-W-S, AE112 is D-F-F-s-r-Y-L-W-S, AE113 is D-N-F-s-r-Y-L-W-S, AE116 is D-Cha-F-s-r-G-Y-L-W-S, AE133 is KGSGG-D-Cha-F-s-r-Y-L-W-S, AE133* is KGSGG-D-Cha-F-s-r-Y-L-W-S, AE134 is KGSGG-D-Cha-F-s-r-Y-L-W-A, AE135 is KGSGG-D-Cha-F-s-r-Y-L-A-S, AE is KGSGG-D-Cha-F-s-r-Y-A-W-S, AE137 is KGSGG-D-Cha-F-s-r-A-L-W-S, AE138 is KGSGG-D-Cha-F-s-a-Y-L-W-S, AE139 is KGSGG-D-Cha-F-a-r-Y-L-W-S, AE145 is KGSGG-D-Cha-F-A-r-Y-L-W-S, AE140 is KGSGG-D-Cha-A-s-r-Y-L-W-S, AE141 is KGSGG-D-A-F-s-r-Y-L-W-S, AE142 is KGSGG-A-Cha-F-s-r-Y-L-W-S, AE143 is KGSGG-D-Chp-F-s-r-Y-L-W-S^{c}, AE144 is KGSGG-D-Cpa-F-s-r-Y-L-W-S^{c}, AE164 is KGSGG-D-F-F-s-r-Y-L-W-S, AE164* is KGSGG-D-F-F-s-r-Y-L-W-S, AE is [D-Cha-F-s-r-Y-L-W-S]2-/3A-K^{c}, AE120* is [D-Cha-F-s-r-Y-L-W-S]2-/3A-K^{c} and AE151 is [r-W-D-Cha-S-L-s-F-Y]2-/3A--K^{c}, or a combination thereof, or a peptide with a sequence homology of at least 80% to any of these peptides.

11. The composition according to claim 1, wherein the receptor is uPAR.

12. The composition according to claim 1, wherein the fluorophore labelled receptor-targeting component is ICG-Glu-Glu-AE105: 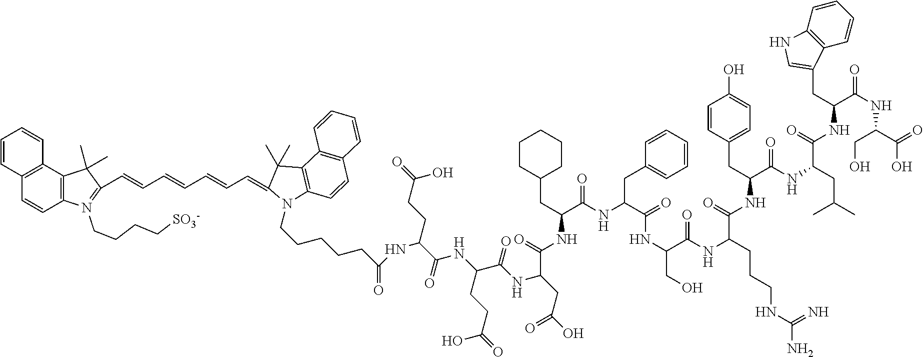
or a pharmaceutically acceptable salt thereof.

13. The composition according to claim 12, wherein the concentration of ICG-Glu-Glu-AE105 is in the range of from 0.1 -10 mg/ml.

14. The composition according to claim 1, wherein the fluorophore labelled receptor-targeting component is ICG-Glu-Glu-AE105: 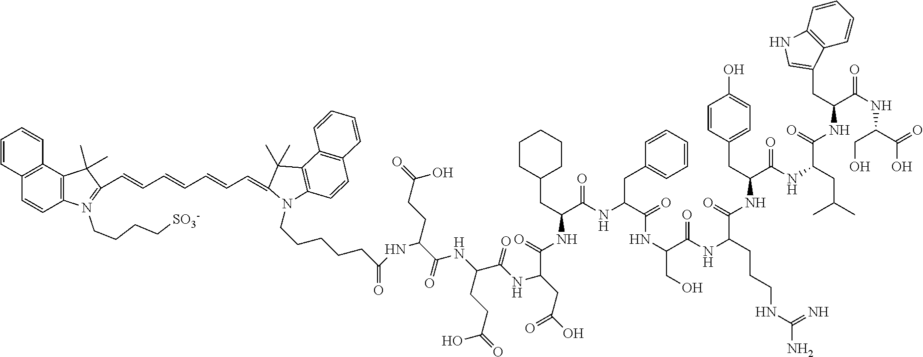
or a pharmaceutically acceptable salt thereof,
wherein the concentration of ICG-Glu-Glu-AE105 is in the range of from 0.1-10.0 mg/ml,
wherein the composition also comprises a buffer in the form of sodium phosphate in concentration of 5-50 mM,
and wherein the composition comprises a cryoprotectant combination of mannitol in a concentration of 10-50 mg/ml and glycine in a concentration of 1-30 mg/ml.

15. The composition according to claim 1, wherein the composition comprises a polysorbate, preferably Polysorbate, more preferably Polysorbate 20 in a concentration of >0.01 wt %.

16-27 (canceled)

28. A lyophilized composition comprising the composition according to claim 1.

29. A method for the production of a composition according to claim 1, wherein said method comprises admixing the fluorophore labelled receptor-targeting component, the buffer, and the surfactant to solubilize the fluorophore labelled receptor-targeting component in the composition.

30. The method according to claim 29, wherein a lyoprotectant is admixed with the composition so that a lyo cake is produced.

31. The method according to claim 29, wherein said method comprises
(i) mixing ICG-Glu-Glu-AE105: 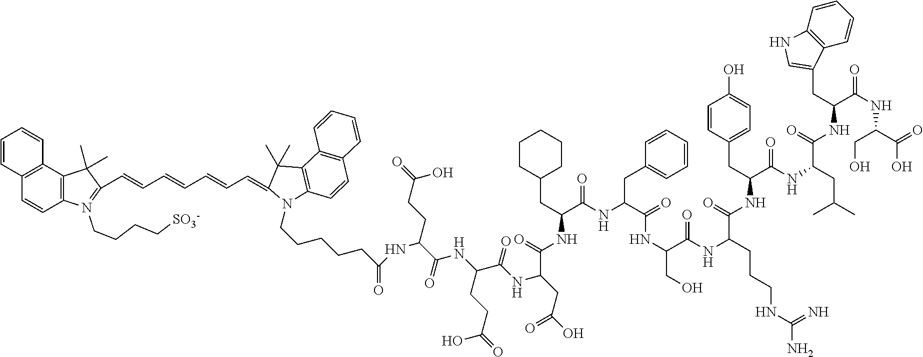
or a pharmaceutically acceptable salt thereof, with sodium phosphate, mannitol and glycine to yield a composition comprising
ICG-Glu-Glu-AE105 in a concentration of 0.1-10.0 mg/ml;
sodium phosphate in a concentration range of 5-50 mM;
mannitol in a concentration range of 10-50 mg/ml;
glycine in a concentration range of 1-30 mg/ml;
Polysorbate 20 in a concentration of >0.01 wt %.
(ii) adjusting the pH of the composition of step (i) to a pH in a range of 6.9-7.9;
(iii) transferring amounts of the mixture from step (ii) equivalent to the desired dosage into a suitable container;
(iv) drying the mixture; and
(v) sealing the container.

32. An optical imaging method comprising the steps of:
(a) administering of a composition according to claim 1 accumulating in a target tissue,
(b) illuminating the target tissue with light of a wavelength absorbable by the fluorophore; and
(c) detecting fluorescence emitted by the fluorophore and forming an optical image of the target tissue.
